(19) United States
(12) Patent Application Publication
(10) Pub. No.: US 2005/0267503 A1
(43) Pub. Date: Dec. 1, 2005

(54) Tissue removal device

(76) Inventor: David L. Hunstad, Rochester, MI (US)

Correspondence Address: BRINKS HOFER GILSON & LIONE, P.O. BOX 10395, CHICAGO, IL, 60610, US

(21) Appl. No.: 10/844,081

(22) Filed: May 11, 2004

(51) Int. Cl. A61B 17/32

(52) U.S. Cl. 606/170

(57) ABSTRACT

The present invention involves a tissue removal device. The tissue removal device comprises a base, a barrel slideably connected to the base, an aspirator system for receiving bone and tissue matter from the barrel, and an actuating lever to move the barrel relative to the base. The base has a distal portion and a track portion extending from the distal portion. The barrel includes a chamber wall having a vacuum chamber formed therein and along the length of the barrel. The vacuum chamber has a distal receiving end configured to cooperate with the distal portion for cutting and removing. The aspirator is in fluid communication with the vacuum chamber to provide a vacuum therein and to receive bone matter from the distal receiving end. The actuating lever is connected to the base and the barrel. The actuating lever is configured to actuate tissue cutting and removing.

### BACKGROUND OF THE INVENTION

The present invention relates to tissue removal devices useful in procedures for spinal surgery.

Spinal surgery, e.g., lumbar, thoracic, or cervical spinal surgery, is a surgical procedure that manufacturers of surgical devices and orthopedic surgeons continually improve. Such procedures may involve removing tissue and/or bone near the spinal cord or spinal nerves. For example, Kerrison™ rongeurs and other tissue removing instruments have been used for tissue removal during spinal surgery. Tissue removal incorporates shearing and cutting of tissue and bone fragments from the vertebrae of a patient. More specifically, a rongeur is typically used to cut, shear, or tear tissue from transverse and spinous processes of the vertebrae to clear an incision area during lumbar, thoracic, or cervical spinal surgery.

Although current tissue removing techniques and instruments are adequate, improvements still may be made. For example, during spinal surgery, an orthopedic surgeon places the distal end of a rongeur at an incision area to cut and shear tissue from the vertebrae of a patient. The surgeon holds the cut tissue in the rongeur which is then removed from the incision wound. The tissue is then manually removed from the rongeur typically by an operative nurse. As further tissue removal is required to clear the incision area, the rongeur is placed back into the incision area for further tissue removal. Accurate placement of the rongeur into the incision area is a concern since the spinal cord and nerves are relatively close. Thus, heightened caution is used when placing the device back into the incision wound.

### BRIEF SUMMARY OF THE INVENTION

One aspect of the present invention provides an improved tissue removal device that eliminates manual bone removal from a tissue removal device. Another aspect of the present invention allows for tissue removing action without shearing and without removal of a rongeur from a point of incision.

In one embodiment, the tissue removal device comprises a base having a distal portion and a track portion extending from the distal portion. The device further comprises a barrel including a chamber wall having a vacuum chamber formed therein and along the length of the barrel. The vacuum chamber has a distal receiving end configured to cooperate with the distal portion for tissue removing. The barrel is slideably connected to the base. The device further comprises an aspirator system in fluid communication with the vacuum chamber to provide a vacuum therein and to receive bone and tissue matter from the distal receiving end. The device further comprises an actuating lever connected to the base and the barrel. The actuating lever is configured to cause the base and the barrel to slide relative to each other and actuate tissue cutting and removal.

Further aspects, features, and advantages of the invention will become apparent from consideration of the following description and the appended claims when taken in connection with the accompanying drawings.

### BRIEF DESCRIPTION OF THE DRAWINGS

FIG. 1 is an environmental view depicting a tissue removal device for cutting bone matter from the vertebrae of a patient;

FIG. 2 is a side view of the tissue removal device in accordance with one embodiment of the present Invention;

FIG. 3 is a perspective break-away view of a distal portion of the tissue removal device in FIG. 2;

FIG. 4 is a cross-sectional break-away view of the tissue removal device taken along line 4-4 in FIG. 2;

FIG. 5 is a perspective break-away view of a distal portion of a tissue removal device in accordance with another embodiment of the present invention;

FIG. 6 is a side view of a tissue removal device in accordance with another embodiment of the present invention; and

FIG. 7 is a side cross-sectional view of the embodiment of the tissue removal device taken along line 7-7 in FIG. 6.

### DETAILED DESCRIPTION OF THE INVENTION

Embodiments of the present invention generally provide an improved tissue removal device useful for clearing a surgical incision area. The tissue removal device allows for continued tissue or bone matter removal at the incision area without requiring removal of the device from the incision area. This embodiment of the present invention eliminates reiterative steps of removing the device from and reinserting the device in the patient, and allows the surgeon to maintain continuous alignment of the device at the surgical incision area.

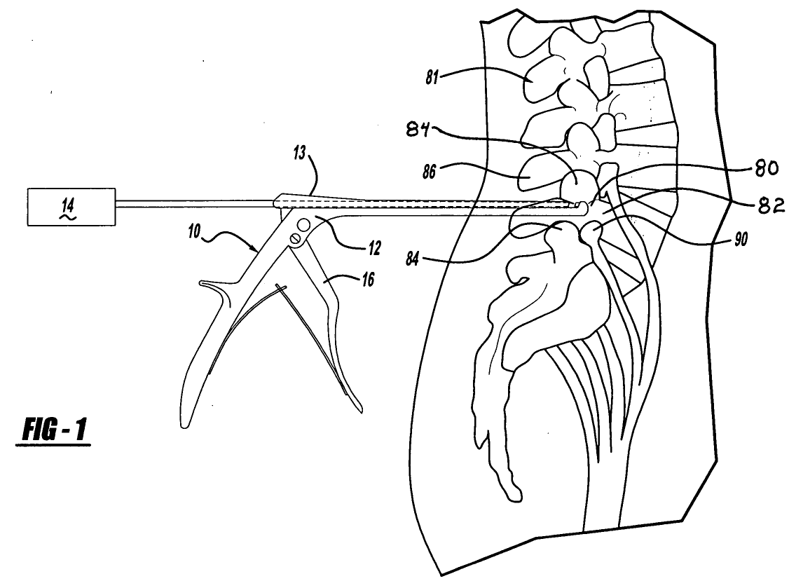

FIG. 1 illustrates an environmental view of a tissue removal device 10 inserted at a surgical incision area 80 for tissue removal from a patient's vertebrae 81. As shown, tissue removal device 10 generally includes a base 12, a barrel 13 slideably connected to the base 12, an aspirator system 14 in fluid communication with the base 12 or barrel 13, and an actuating lever 16 connected to the base 12 and the barrel 13 causing the base and the barrel to slide relative to each other and actuate the aspirator system 14. The aspirator system 14 provides a suction through the base 12 or barrel 13 to facilitate removal of cut tissue from the vertebrae 81.

As shown in FIG. 1, tissue removal device 10 is inserted at the surgical incision area 80 within vertebrae 81. In this example, the surgical incision area 80 is located at the lumbar vertebrae 82 of a patient. During a surgical procedure on spinal cord root 90, device 10 is used to clear a surgical path through the vertebrae 81. In this embodiment, a portion of the transverse process 84 extending from the spinous process 86 may be cleared using tissue removal device 10. As will be discussed in greater detail below, tissue removal device 10 may be maintained in surgical incision area 80 without removal from the area 80, while cutting and suctioning tissue therefrom. Thus, this embodiment of the present invention maintains alignment of the device in the surgical incision area 80 and continuous cutting and removing of tissue from the vertebrae 81. It is to be understood that tissue removal device 10 may be used in other areas of the body of a patient.

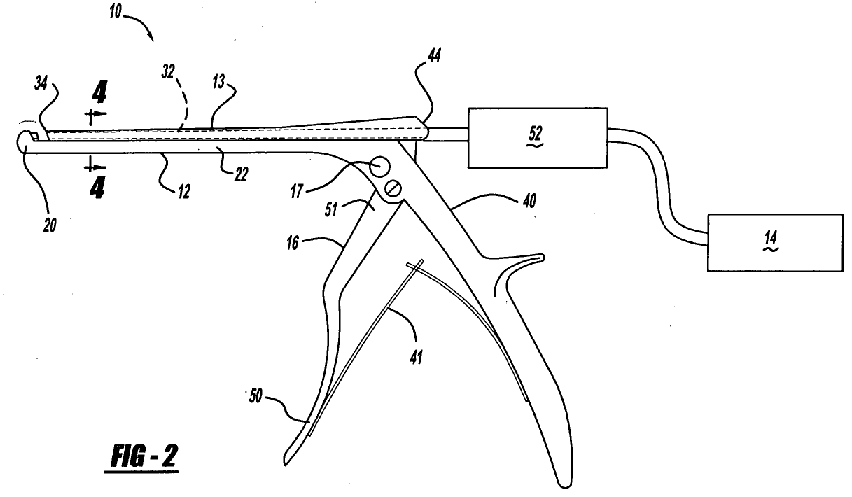

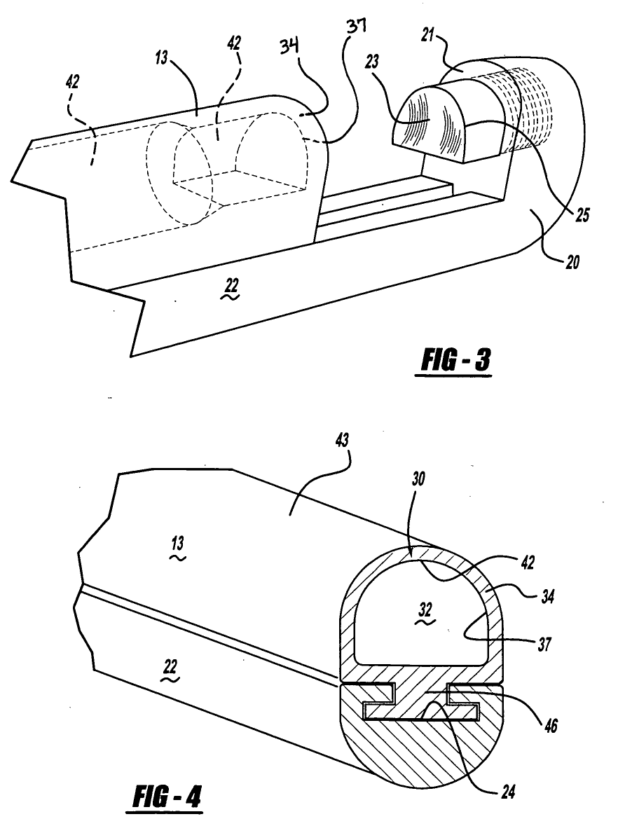

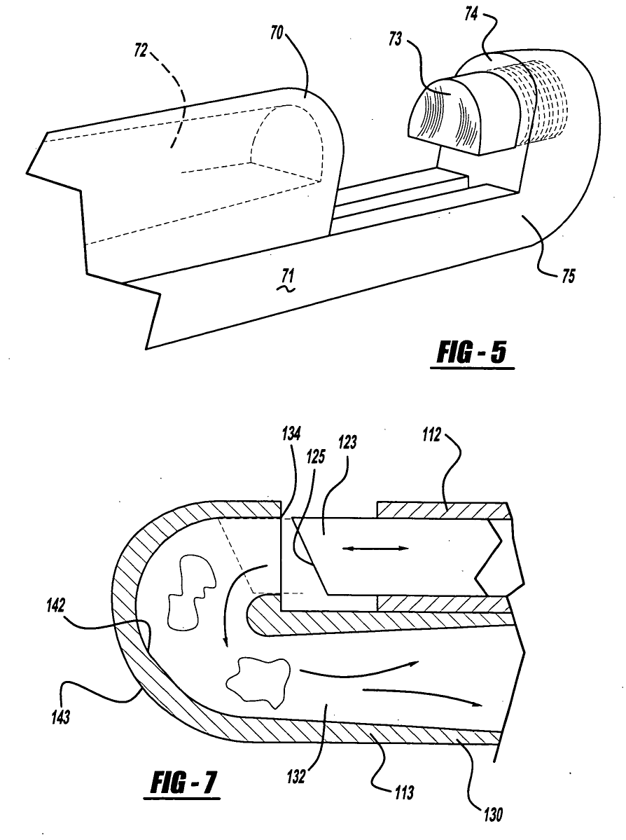

FIGS. 2 and 3 illustrate the base 12 having a distal portion 20. The distal portion 20 includes a distal flange 21 and a distal plunger 23 extending proximally from the distal flange 21. As shown, the distal plunger 23 takes on a shape of a semi-circle, but may take on any other suitable shape without falling beyond the scope or spirit of the present invention. Preferably, the distal plunger 23 has sharpened outer edges 25 to facilitate cutting tissue and bone. The base 12 further includes a track portion 22 extending from the distal portion 20. The track portion 22 preferably has a planar surface on which a sliding groove 24 is formed. As shown, the base 12 further includes a proximal handle 40 extending from the track portion for leverage during tissue removal.

FIGS. 2 and 4 depict the barrel 13 including a chamber wall 30 having a vacuum chamber 32 formed therein and extending along the length of the barrel 13. As shown, the chamber wall 30 has inner and outer surfaces 42 and 43. The inner surface 42 is formed along the length of the barrel 13, defining the vacuum chamber 32. The chamber wall 30 extends from a distal receiving end 34 to a proximal exiting end 44. As shown in FIGS. 2-4, the cross-sectional area of the vacuum chamber 32 increases from the distal receiving end 34 to the proximal exiting end 44.

The distal receiving end 34 of the vacuum chamber 30 is configured to cooperate with the distal portion 20 for tissue removal. In this embodiment, the distal receiving end 34 receives the distal plunger 23 and engages the distal flange 21 for tissue cutting and removing action. As shown, the distal receiving end 34 has sharpened inner edges 37 and mates with the sharpened outer edges 25 of distal plunger 23 for cutting action. At an incision area of a patient, device 10 is configured to engage bone or tissue between the distal receiving end 34 and the distal plunger 23 for cutting. As the distal receiving end 34 receives the distal plunger 23, bone and tissue that are engaged therebetween are cut and plunged into vacuum chamber 30 for removal.

As shown in FIGS. 3 and 4, the barrel 13 is slideably connected to the track portion 22. The barrel 13 has a tongue 46 extending from the outer surface 43 of the chamber wall 30 and is formed along a portion of the length of the barrel 13. The tongue 46 mates and cooperates with the groove 24 to allow the barrel to slide along the track portion 22.

The aspirator system 14 is configured to be in fluid communication with the vacuum chamber 32, providing a suction therein and receiving bone matter from the distal receiving end 34. The aspirator system 14 has a manual activation mechanism for activating the aspirator system 14 to receive bone matter from the vacuum chamber 32. The manual activation mechanism may include an activation switch disposed on the device 10 to activate the aspirator system 14. The aspirator system 14 is preferably connected to the proximal exiting end 44 to receive bone matter. Preferably, a suction switch 17 which may be located on the actuating lever 16, on barrel 13 or proximal handle 40 is configured to allow the surgeon to selectively activate the aspirator system 14. For example, the suction switch 17 may be connected to a valve door separating the vacuum chamber 32 and aspirator system 14 within the barrel 13.

As shown in FIG. 2, a filter trap 52 is in fluid communication with the distal receiving end 34 and the aspirator system 14. The filter trap 52 is disposed between the distal receiving end 34 and the aspirator system 14 for filtering bone and tissue matter from the vacuum chamber 32.

In this embodiment, the actuating lever 16 is connected to the base 12 and the barrel 13. Generally, the actuating lever 16, when moved, slides the barrel 13 along the track portion 22. More specifically, FIG. 2 illustrates the actuating lever 16 having first and second portions 50 and 51. The first portion 50 is connected to the proximal handle 40 by spring 41. The second portion 51 is pivotally connected to the proximal handle 40 of the base 12 and engages the barrel 13 so that, as the second portion 51 pivots relative to the proximal handle 40, the barrel moves along the track portion 22. Spring 41 is connected to the first portion 50 and the proximal handle 40 to bias the first portion 50 from proximal handle 40, defining a resistance therebetween. The actuating lever 16 is configured to move relative to the proximal handle 40, sliding the barrel 13 for and aft along the track portion 22 for tissue cutting and removal. Thus, spring 41 provides bias resistance to a surgeon, requiring movement of the first portion 50 toward proximal handle 50 for tissue cutting action. In use, the surgeon squeezes or applies pressure to the proximal handle 40 and first portion 50 together to initiate cutting. As needed, the surgeon activates switch 17 to suction bone matter therefrom.

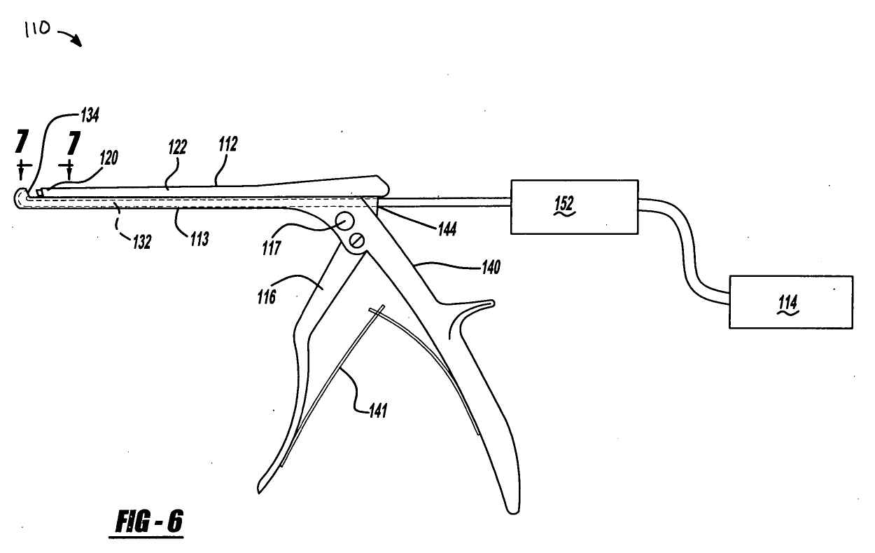

FIGS. 6 and 7 illustrate a tissue removal device 110 in accordance with another embodiment of the present invention. The device 110 includes similar components as the components of device 10 discussed above. However, in this embodiment, the device 110 includes a base 112 slidably movable relative to a barrel 113. More specifically and referring to FIG. 6, device 110 comprises a base 112 having a distal portion 120 and a track portion 122 extending from the distal portion 120. As shown, the base 112 further includes a plunger 123 distally extending from the distal portion 120 for tissue cutting. The plunger 123 preferably has sharpened outer edges 125 for enhanced cutting action.

The device 110 further comprises a barrel 113 including distal receiving end 134 and a chamber wall 130 extending therefrom to define a vacuum chamber inner surface 132. As shown, the chamber wall 130 extends along the length of the barrel 113 and includes inner and outer surfaces 142 and 143. The inner surface 142 is formed along the length of the barrel 113, defining the vacuum chamber inner surface 132.

In this embodiment, chamber wall 130 distally extends from the distal receiving end 134 and is formed to curve proximally to a proximal exiting end 144. FIG. 7 illustrates that the distal receiving end 134 is configured to cooperate with the plunger 123 for tissue cutting similar to the plunger 23 and distal receiving end 34 in the first embodiment discussed above. As shown, the distal receiving end 134 receives tissue that has been cut and distally plunged by the plunger into the vacuum chamber inner surface 132. The tissue may then be suctioned proximally through the barrel 113 as discussed above. Although not drawn to scale, it is to be understood that the cross-sectional area of the barrel increases relative to cross-sectional area at the distal receiving end 134. This may be accomplished by a step change or continual increase in area toward the proximal exiting end 144. As a result, the likelihood of tissue or bone fragments caught in the vacuum chamber 132 is reduced.

Similar to the device 10 discussed above, the device 110 further comprises an aspirator system 114 in fluid communication with the vacuum chamber inner surface 132 to provide a vacuum therein and to receive bone matter cut at the distal receiving end 134. In this embodiment, barrel 113 further includes a proximal handle for leverage during tissue removal. Moreover, an actuating lever is preferably connected to the base and the barrel to actuate tissue cutting and removal similar to the embodiment mentioned above.

As any person skilled in the art will recognize from the previous description and from the figures and claims, modifications and changes can be made to the preferred embodiment of the invention without departing from the scope of the invention as defined in the following claims.

1. A tissue removal device comprising:
a base having a distal portion and a track portion extending from the distal portion;
a barrel including a chamber wall having a vacuum chamber formed therein and along the length of the barrel, the vacuum chamber having a distal receiving end configured to cooperate with the distal portion for tissue cutting and removing, the barrel being slidably connected to the base;
an aspirator system in fluid communication with the vacuum chamber to provide a vacuum therein and to receive bone matter from the distal receiving end; and
an actuating lever connected to the base and the barrel, the actuating lever configured to cause the base and the barrel to slide relative to each other and actuate tissue cutting and removal.

2. The device of claim 1 wherein the base further includes a proximal handle extending from the track portion for engaging with the distal portion for tissue removing, the track portion having a groove formed thereon.

3. The device of claim 1 wherein the chamber wall has inner and outer surfaces, the inner surface defining the vacuum chamber formed along the length of the barrel.

4. The device of claim 3 wherein the chamber wall includes a proximal exiting end extending from the distal receiving end.

5. The device of claim 4 wherein the barrel has a tongue extending from the outer surface of the chamber wall and formed along a portion of the length of the barrel, the tongue cooperating with the groove to slide the barrel along the track portion.

6. The device of claim 4 wherein the aspirator system is connected to the proximal exiting end to receive bone matter.

7. The device of claim 2 wherein the actuating lever has first and second ends, the first portion being connected to the proximal handle and the second end being connected to the barrel, the actuating lever being configured to move relative to the proximal handle to slide the barrel along the track portion for engaging with the distal portion for tissue removing.

8. The device of claim 4 wherein the chamber wall extends from the distal receiving end and the vacuum chamber has increased diameter from the distal receiving end.

9. The device of claim 4 wherein the chamber wall extends from the distal receiving end and the vacuum chamber has a larger diameter than the distal receiving end.

10. The device of claim 4 further comprising a filter trap in fluid communication with the distal receiving end and the aspirator system, the filter trap being disposed between the distal receiving end and the aspirator system for filtering bone matter from the vacuum chamber.

11. The device of claim 1 wherein the aspirator system has a manual activation mechanism for activating the aspirator system to receive bone matter from the vacuum chamber.

12. The device of claim 11 wherein the manual activation mechanism includes an activation switch disposed on the device to activate the aspirator system.

13. The device of claim 1 wherein the distal portion includes a distal flange and a distal plunger extending from the distal flange, the distal receiving end being configured to receive the distal plunger and to engage the distal flange for tissue removing action.

14. The device of claim 1 wherein the track portion has a groove formed thereon.

15. A tissue removal device comprising:
a base having a distal portion, a track portion extending from the distal portion, and a proximal handle extending from the track portion, the track portion having a groove formed thereon;
a barrel including a chamber wall having inner and outer surfaces, the inner surface defining a vacuum chamber formed along the length of the barrel, the vacuum chamber having a distal receiving end and a proximal exiting end, the distal receiving end being configured to cooperate with the distal portion for cutting and passing bone matter therethrough, the barrel having a tongue extending from the outer surface of the wall and formed along a portion of the length of the barrel, the tongue cooperating with the groove to slide the barrel along the track portion;
an aspirator system connected to the proximal exiting end and in fluid communication with the vacuum chamber to provide a vacuum therein and to receive bone matter therefrom; and
an actuating lever having first and second ends, the first portion being connected to the proximal handle and the second end being connected to the barrel, the actuating lever being configured to move relative to the proximal handle to slide the barrel along the track portion.

16. The device of claim 15 wherein the chamber wall extends from the distal receiving end and the vacuum chamber has increased diameter from the distal receiving end.

17. The device of claim 15 wherein the chamber wall extends from the distal receiving end and the vacuum chamber has a larger diameter than the distal receiving end.

18. The device of claim 15 further comprising a filter trap in fluid communication with the distal receiving end and the aspirator system, the filter trap being disposed between the distal receiving end and the aspirator system for filtering bone matter from the vacuum chamber.

19. The device of claim 15 wherein the aspirator system has a manual activation mechanism for activating the aspirator system to receive bone matter from the vacuum chamber.

20. The device of claim 19 wherein the manual activation mechanism includes an activation switch disposed on the device to activate the aspirator system.

21. A method of tissue removal from an incision area of a patient, the method comprising:
placing a cutting device at the incision area;
cutting tissue at the incision area of the patient; and
suctioning the tissue at the incision area while maintaining placement of the cutting device at the incision area.
